(19) United States
(12) Patent Application Publication
(10) Pub. No.: US 2020/0282087 A1
(43) Pub. Date: Sep. 10, 2020

(54) Heavy Traffic Sanitization

(71) Applicant: Lightcraft Technologies, LLC, Manassas, VA (US)

(72) Inventors: Rohullah Latif, Fullerton, CA (US); Jacob Daniel Conner, Rogersville, MO (US); Michael Keith Blankenship, Bristow, VA (US)

(73) Assignee: Lightcraft Technologies, Manassas, VA (US)

(21) Appl. No.: 16/879,753

(22) Filed: May 20, 2020

(51) Int. Cl. A61L 2/00; A61L 2/26

(52) U.S. Cl. CPC A61L 2/0047; A61L 2202/11; A61L 2/26

(57) ABSTRACT

The present invention includes light sanitization having a dosage duration that is informed with cues and made more efficient through the use of sanitization enabling components. For example, applying a bidirectional stream of airflow in concert with sanitization enhances the surface area exposure.

### FIELD OF THE INVENTION

The present invention relates to the field of sanitation and hygiene and more specifically to the field of choke-point pathogen destruction.

### BACKGROUND

Pathogens, such as viruses, bacteria, and fungi, are responsible for numerous diseases or infections, including some very dangerous and potentially fatal diseases and infections, that affect humans, animals, and plants. Environments, such as health-care environments (e.g., hospitals) and restaurants, are particularly susceptible to the transmission or spread of such pathogens. Indeed, healthcare associated infections (HAIs), which are caused by pathogens, such as Methicillin-resistant Staphylococcus aureus (MRSA), Clostridium difficile (C. difficile), and Mycobacterium tuberculosis, transmitted through, for example, the air, person-to-person contact, and skin shedding in healthcare environments, are an increasingly dangerous problem for the healthcare industry. According to the Center for Disease Control and Prevention, HAIs cause at least 1.7 million illnesses and 99,000 deaths in acute care hospitals in the U.S. alone every year. Pathogens can also serve to spoil food products (e.g., fruits, vegetables) and result in the loss of goods and raw materials in various industrial processes, for example chemical processing, brewing and distillation, food packaging, and other processes that require non-contaminated environments. Significant resources have already been committed to preventing and controlling pathogens in these environments, but to this point, these resources have not yielded the desired results. Some existing methods of pathogen control, e.g., those involving hygiene, have proven to be labor-intensive, difficult to monitor, and, most importantly, of limited effectiveness (e.g., are only temporarily effective, only deactivate some pathogens). Attempts to utilize light emission sources to control pathogens and unwanted chemicals have yet to find widespread acceptance based on multiple deficiencies, including the principal that light cannot sanitize that which it cannot reach. The present invention is an attempt to expand the efficacy of light-based sanitization, even in heavy traffic corridors.

### SUMMARY

The present invention is directed to an electromagnetic spectrum disinfectant device, system, and processes for using the same. The present invention includes a designated area for a user to occupy. A preferred embodiment of the present invention includes a bounded tunnel with a focal point. A focal point for purposes of the present invention is a position in the designated area towards which cleansing implements of the present invention may be oriented. By cleansing implements, it is meant light sources and a components to allow enhanced access by the light sources.

The light source of the present invention can include any light emission component capable of delivering a light stream sufficient to neutralize a pathogen or alter a chemical. Because the present invention applies to heavy traffic areas of various, myriad types, the light source may be selected based on the type of chemical/pathogen desired to be neutralized. The light source is adapted to emit an electromagnetic frequency of light effective for order-of-magnitude destruction of predetermined pathogens, or alteration of a predetermined chemical (or class thereof), for a dose duration. In the preferred embodiment, the present invention is principally useful in sanitizing organisms. Because organisms have multiple nooks and crannies, and perhaps of greater significance have coverings, e.g. clothing, with nooks and crannies, the light source should be emitted from multiple vantage points. For humans, it is preferred that a medial point be established demarcating a point below which light should be emitted and above which light should be emitted.

As discussed above, a significant deficiency in light-based cleansing is light's inability to “flow” around or penetrate a wide of materials. Light contacting a covered organism may only have access to 80-95% of the organisms exterior surface. The present invention overcomes this deficiency by applying environmental agitation to the focal point, and perhaps beyond, to expose otherwise unavailable surface. The preferred agitation is delicate agitation sufficient to jostle clothes, but not to re-orient them. Furthermore, the preferred agitation include at least two vectors of agitation such that there is not simply a stream of physical contact, such as would be created by a single, stationary fan. The preferred basis of agitation is air-based.

These aspects of the invention are not meant to be exclusive. Furthermore, some features may apply to certain versions of the invention, but not others. Other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is a plan view of the device of the present invention.

FIG. 2 is a revealed view of the device of the present invention.

FIG. 3 is a perspective view of the device of the present invention.

FIG. 4 is a revealed, perspective view of the device of the present invention.

FIG. 5 is a cutaway, perspective view of the device of the present invention.

FIG. 6 is a cutaway, perspective view of the device of the present invention.

FIG. 7 is a plan view of the device of the present invention.

FIG. 8 is a revealed view of the device of the present invention.

FIG. 9 is a perspective view of the device of the present invention.

FIG. 10 is a perspective view of the device of the present invention.

FIG. 11 is a perspective view of the device of the present invention.

FIG. 12 is a top plan view of the device of the present invention.

FIG. 13 is a view of the method of the present invention.

FIG. 14 is a top plan view of the device of the present invention.

FIG. 15 is a top plan view of the device of the present invention.

FIG. 16 is a view of method implementation steps of the present invention.

FIG. 17 is a view of method implementation steps of the present invention.

### DETAILED DESCRIPTION

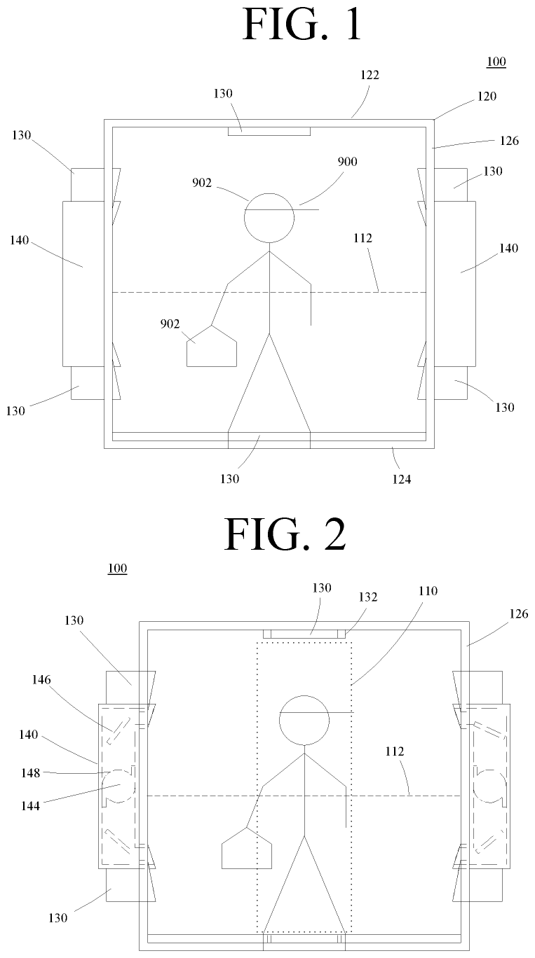

Referring first to FIG. 1, a basic embodiment of the electromagnetic spectrum disinfectant device 100 is shown. A mass traffic version of the device 100 includes a bounded tunnel 120 with a centralized focal point 110 for a user 900 to occupy. The significance of the tunnel 120 is that it creates a choke point to ensure that users are allowed into the device in meaningful way that allows sufficient disinfecting of the users. As can be seen in other embodiments of the present invention, the choke point can be created via alternative structures, but the common thread is that a user can receive a dose of electromagnetic radiation in a way wherein other users do not block the path of the radiation. The user is bounded by light emission sources and the structures that bear them. Bounded for purposes of the present disclosure means that the user encompassed in at least one dimension by structure, which can be any (or more) of side-to-side, up-and-down, or front-to-back.

The tunnel 120 has a floor 122, ceiling 124, and sidewalls 126. Each of these surfaces can exist for purposes of support of components or to retain emission/light sources 130. These surfaces if performing none of the aforementioned functions can be omitted. In the preferred version of the tunnel 110, there is a floor that includes a partially transparent floor 124 for the inclusion of emission sources 130 or outlet for emission sources. The sidewalls 126 of the tunnel 110 support principal emission sources 130 as well as disinfectant enablers 140. By disinfectant enablers, it is meant a component, that although is not inherently itself a basis for disinfection, acts as an accessory to aid the emission sources 130 in their disinfectant function. The primary version of disinfectant enablers includes environmental agitation units 140.

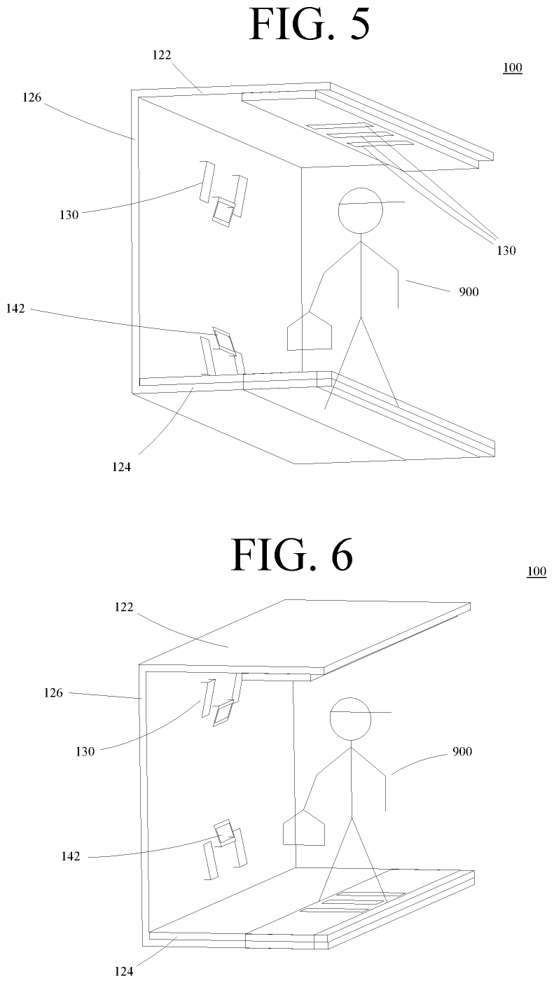

As shown in FIGS. 1-6, the preferred placement of the emission sources 130 is best discussed with reference to a medial point or line 112 that is derived from understanding the user for whom the disinfection is meant. As discussed the emission source 130 is adapted to emit an electromagnetic frequency of light effective for order-of-magnitude destruction of predetermined pathogens, or alteration of a predetermined chemical (or class thereof), for a dose duration. Because organisms have multiple nooks and crannies, and perhaps of greater significance have coverings, e.g. clothing, with nooks and crannies, the emission source should be emitted from multiple vantage points. For humans, it is preferred that the medial point 112 be established demarcating a point below which light should be emitted and above which light should be emitted. A preferred medial point is based on organism body characteristics, such as height. A sensible medial point 112 is some fraction of the organism's height, width, etc. Alternatively, the medial point 112 can correspond to organism features that define substantial shade points, e.g. shoulders or thighs.

Emission sources may placed in configurations that allow the user 900 to be exposed to the light from the top down, bottom up, left to right, and right to left, thus exposing the subject up to light emission from many, varied angles. As the quantity of lights, or light sources, increases, so too does the likelihood of exposing more surface area. Fewer lights can be used if the light sources are moving such as on a robotic gantry that rotates or moves to continuously expose new surface area. Furthermore, the lights can be fixed and mirrors or reflectors can be moving or pivoting such as a mirror ball or in the case of lasers a galvanometer scanner.

Light emission sources of the present invention include excimer lamps, flash lamps, arc lamps, Hyrdogen lamps, and laser sources, either in isolation or combination. Excimer or Exciplex lamps are quasimonochromatic light sources operating over a narrow range of wavelengths dependent on the gas fill of the lamp in the ultraviolet (UV) and vacuum ultraviolet (VUV) spectral regions. Operation of an excimer lamp is based on the formation of excited dimers (excimers), which spontaneously transiting from the excited state to the ground state result in the emission of UV-photons. The spectral fingerprint of excimer lamp radiation is specified by a working excimer molecule. They are narrow band, operating from emission lines from excited dimers such as (Molecular Chlorine, KrBr, ArCl, ArF, KrF, KrCl, molecular fluorine/F2, Xe Excimer, Kr Excimer, Ne Excimer). Two preferred dimers include: KrBr that emits at 207 nm and KrCl that emits at 222 nm. There is some support for the idea that 207-222 nm is effective in destroying pathogens without harming human skin or eye. It is long enough in wavelength that it does not produce a lot of ozone, nor it is as strongly absorbed by oxygen, while yet being short enough that it does not penetrate deep into human tissue.

Flash lamps are lamps that produce a pulsed light ranging in milliseconds to picoseconds. Flash lamps are a relatively cost-effective, low-power solution that allow low cost, pulsed emissions that result in high peak power in short pulses. It can thermally & photochemically disrupt biological pathogens and contaminants. It is broadband continuous emission spectra with peaks in Kr and Xe that can be shifted towards the UV by increasing the Amps/Sq/Cm above 4500 AMps/Sq/Cm current density. That can further be filtered with a High Pass Filter filter (225 nm or shorter) or Band Pass Filter from 207-222 nm. Filtering can be accomplished by reflective mirrors or absorptive filters.

Arc lamps, e.g. Xe/Kr/Metal Halide, produce continuous waves that generate broad spectrum with strong peeks for XE & Kr. Filter or mirrors can also be added to ensure spectrum is limited to 207-222 nm. Arc lamps are an alternative to excimer lamps for continuous wave UV output.

Magnetron radiofrequency excited flat panel excimer lamps can provide emissions at an effective range of 207-222 nm. Instead of a discharge barrier (capacitively coupled) lamps, RF energy is directed to the lamp via waveguide. High power and low cost. It can be used with excimer gases (KrCl 222 nm or KrBr 207 nm). Hydrogen or Deuterium emission sources can provide another UV source at relatively low power. This operate using a hollow cathode discharge.

Lasers may be light emission sources for the present invention. Harmonic lasers that utilize sum/difference frequency generation, or second through fifth harmonic generation, may be utilized. Light may be masked, or some other means of physically manipulating the beam characteristics. Light beam expansion may be utilized to expand the beam to a substantial width in order to permit beneficial surface area contact of a user. For example, a laser beam may be expanded to four inches but the intensity or irradiance must be adjusted in relation to time and power. For every doubling of area of the beam diameter there is a drop-in intensity to the fourth power. The fluence is an exposure dose of an area integrated over time. Larger beams will have a lower watts/cm\hat{ }2 for a given light source and this can be accounted for by increasing the exposure time or increasing the power of the light source. Excimer lasers may be utilized that allow access to deep and far UV spectra. The beam may be scanned with mirrors onto surfaces. Ion lasers such as He-Silver metal vapor may be utilized.

The particular electromagnetic spectrum characteristics of the light emission source may be many and varied as such suit the particular use to which the invention may be applied. In instances where a pathogen of known characteristics is the target, then the light emission sources may be tuned, or replaced, to apply EM spectra suitable for the destruction of such pathogen. In instances whereby a chemical is desired to be destabilized or destroyed, the particular EM spectra known to cause such destabilization/destruction may be utilized. The EM spectra, intensity, etc. are frequently known and subject, simplistically, to mathematical treatment. For example, in U.S. Pat. No. 10,456,485, the disclosure of which is hereby incorporated by reference, the disclosure describes in significant detail the characteristics of EM spectra that successfully acts upon pathogens.

The '485 patent explains that a lighting device that provides or delivers (e.g., outputs, emits) at least 3,000 mW (or 3 W) of disinfecting light, which has a wavelength in the range of approximately 380 nm to approximately 420 nm, and more particularly between 400 nm and 420 nm, to the environment, as it will be appreciated that doses of light having a wavelength in this range but delivered at power levels lower than 3,000 mW are generally ineffective in deactivating dangerous pathogens. A lighting device may, for example, provide or deliver 3,000 mW, 4,000 mW (or 4 W), 5,000 mW (or 5 W), 6,000 mW (or 6 W), 7,000 mW (or 7 W), 10,500 mW (or 10.5 W), or some other level of disinfecting light above 3,000 mW. A light source that also provides or delivers levels of disinfecting light such that any exposed surface within the environment has or achieves a desired, minimum power density while the lighting device is used for deactivation, thereby ensuring that the environment is adequately disinfected. This desired, minimum power density is the minimum power, measured in mW, received by any exposed surface per unit area, measured in cm squared. When measured or determined over time (the period of time over which the lighting device is used for deactivation), this minimum power density within the applicable bandwidth of visible light may be referred to, as it is herein, as the minimum integrated irradiance, measured in mW/cm². The minimum integrated irradiance of the disinfecting light provided by the light source, which in this example is measured from any exposed surface or unshielded point in the environment that is 1.5 m from any point on any external-most luminous surface of the lighting device but may in other examples be measured from a different distance from any external-most luminous surface, nadir, any unshielded point in the environment, or some other point, is generally equal to at least 0.01 mW/cm². The minimum integrated irradiance may, for example, be equal to 0.02 mW/cm², 0.05 mW/cm², 0.1 mW/cm², 0.15 mW/cm², 0.20 mW/cm², 0.25 mW/cm², 0.30 mW/cm², or some other value greater than 0.01 mW/cm².

According to the Materion Corporation, The market for UV-C disinfection is blossoming, driven partly by recent outbreaks and fear of viruses and bacteria such as SARS, MERS, MRSA, Ebola, norovirus and C-DIFF. UV light emitting diodes (“LED”) can play a useful role in preventing infectious disease. They may be used to make water potable, replace chlorine as a water disinfectant in swimming pools, kill germs in clothes washers and dishwashers, kill airborne germs in air purifiers and HVAC systems, and disinfect surfaces in hospitals, kitchens, schools, offices and nursing homes. UV-C LED products are already available for high-end applications like industrial water purification, but there is a strong push to reduce the cost of the LED chips in order to address the very large consumer market for disinfection.

Materion goes on to explain that a challenge in packaging UV-C LEDs is the window mounted atop the LED package. Nearly all organic materials absorb UV-C radiation, so the same “glob top” silicones used atop visible light, UV-A and UV-B LEDs are inappropriate for UV-C LEDs. The only two practical window materials for UV-C LEDs are high purity silica glass (fused silica SiO2) and sapphire (Al₂O₃). Another unique challenge with UV-C LEDs is their low efficiency; conventional UV-C LEDs have an efficiency <15%, so it is very important to include an anti-reflective (AR) coating on both faces of the window in order to maximize photon emission from the package.

Although the characteristics of the light wave tasked to denature/destroy/modify pathogens and chemicals has been dealt with in some detail, the art has utilized relatively less thought in the fundamental differences between light and other cleansing media. Accordingly, the present invention applies disinfectant enabling devices 140 of multiple shapes and forms. As shown in FIGS. 1-6, a preferred disinfectant enabling device 140 includes an environmental agitator. The agitators 140 of FIGS. 1-6 include mechanisms that physically disturb the environment immediately surrounding the user to aid the light emission sources in disinfecting the user 900. The agitator 140 of the present invention includes an air motivator 144 with access to a plenum 148 that shunts air in the direction of baffles 146 or other interruption airflow components. The baffles 146, which may be either stagnant or moving, affect the airflow such that the airflow oscillates.

The oscillating airflow may be path-matched to work in tandem with an agitation device 140 on an adjacent sidewall such that a sanitization area 110 in between the agitators 140, in an advantageous position in the device 100, receives airflow in more than one direction—without one airflow pathway in a constant state of overpowering the other. The natural position for the sanitization area 110 of the device 100 is the exact center (and surrounded space) of the device 100. It is not preferred that the agitators 140 work such that one is always dominant in relation to an opposite agitator; otherwise, a scenario similar to that of a simple blower would be created. The agitation of the present invention works similar to that of a (relatively dry) washing machine. The clothes and other articles 902 worn by a user or not simply accessed by the light emission sources, but rather clothes and other articles 902 with motionable surfaces have as much of the surface as is feasible to be accessed by light waves. For example, a woman's blouse may be fabricated of a lightweight material such that crinkling reveals only 95% of the blouse at any one time. As that woman walks about in a normal environment shifting winds or HVAC pressures move and alter the surface area of the blouse most readily available to the outside environment. In an environment whereby no agitation is achieved, light sanitization occurs only on those areas that happen to be available and accessible at that moment in, or around, the device. Multidirectional agitation assures that not only the surfaces accessible at the moment of entering the device contact light, but also those surfaces that were accessible at any time during the wearing of the garment.

Having an agitator that merely provides one direction of airflow will not achieve this objective because doing so only creates a second dominant state of surface area exposure. However, a state of agitation whereby a force/stagnant airflow or positive/negative pressure environment is created ensures that a significant amount of the any garment is exposed to light waves. The preferred agitators are tuned such that within whatever area is determined to be the designated sanitization area 110, all areas inside the area 110 are susceptible to a force and counter-force situation described earlier as the force/stagnant and positive/negative situation. Positions outside of this area, such as may exist immediately adjacent to one or other agitator, may result in airflow power insurmountable by the more distant agitator and thus a multi-directional agitation does not occur. Preferred rate of agitation and wind force can be described according to the Beaufort Wind Scale 2-5. Select Beaufort Wind Scale indicates as follows; 1: Smoke drift indicates wind direction, still wind vanes; 2: Wind felt on face, leaves rustle, vanes begin to move; 3: Leaves and small twigs constantly moving, light flags extended; 4: Dust, leaves, and loose paper lifted, small tree branches move; 5: Small trees in leaf begin to sway; 6: Larger tree branches moving, whistling in wires.

Because in light sanitization, the position and orientations of the user are of much greater import than in liquid/chemical sanitization, the position and orientations that a user should effect should be communicated to the user. The communications can be express, however, over communications are time consuming and require a common language basis between the present invention and the user 900. It is preferred that audio or visual cues are provided to the user indicative of preferred positions and orientations. It is further preferred that such cues are inferential cues, rather than explicit cues, such that information concerning position and orientation must be inferred from the cue rather than directly on point. For example, an audio voice explaining to a user to maintain a position within a designated area of the device is an express cue, while a buzzer that sounds when a user leaves the designated area would be an inferential cue.

Audio Cues and Alternatives Path Completion Cues. In a first version of audio cues, a path cue exists that guides a user 900 in the rate at which s/he should complete the device exposure pathway. Electromagnetic radiation is common; however, because EM radiation is nonetheless energy exposure is preferably kept to a minimum, it is preferred that light emission sources are initiated and terminating according to a duration calculated to be effective at supplying a sanitization dose for the most minimal duration. Accordingly a dosage range may be calculated based on the distance of the user from the light emission sources (here, the significance of the designated sanitization area is shown), the intensity of the light emission sources, and the time—as well as extraneous factors such as age, weight, sensitivity (based, for example, on known instances of melanoma, cancer, or other health issue). In a time-based solution to the present problem, a user 900 is placed in the vicinity of a light emission source and the light emission source is activated for that dosage time period. However, it is a feature of the present invention to provide a visual cue that indicates the mechanical basis by which a user maintains exposure to the light emission sources based on their position and orientation. For example, for light emission sources having a calculated dosage exposure period of ten seconds being deployed in a device having a hallway of five meters can create audio cues related to steps determined to cover a rate of 0.5 m/s. According a beat intended to mimic footstep rates suitable for an average person, or some other calculation adapted to be more suited to the person traveling through the device 100, can be applied through speakers. The backbeat audio cue creates a subliminal sensation that subconsciously urges the user 900 to step in time with the backbeat.

Other audio cues can be used to laterally position the user 900 within the designated sanitization area 110 of the device 100. For example, if a device 100 has a designated sanitization area 110 of one meter in the immediate center of the device hallway, a position indicator 170 provides a position and possible velocity of a user 900 with the device and initiates a buzzing cue that remains in effect for so long as the user maintains an unfavorable position. Furthermore, because the dosage period is calculated for a best mixture of efficacy (maximized) and exposure (minimized), velocities that may result in prolonged or maximized exposure can be detected and result in an audio cue. For example, stopping within the device can result in an inferential cue relating in inaction. A velocity that may result in an insufficient dosage period can result in an audio cue indicative of excessive speed or indicative of the need to slow. Such cues would by nature be different notwithstanding that the same result is desired. In certain cultures, a tonal cue with a rapid beat may indicate to a user the need to speed up rather than indicate that the existing speed is already too high. Furthermore, the tonal cue that is the baseline, for example the previously-discussed backbeat, may be altered to comport with the effective dosage period. For example, a tonal backbeat may be calculated to apply to a standard rate of footstep progress in a hallway, and as a user exceeds the rate the tonal backbeat rate may be decreased to as to cue a slowdown to footstep progress, and vice-versa.

Instead of directing the user using visual or audio cues, the machine may adjust the dosage/power independent of audio or visual cues.

Visual Cues and Alternatives Visual cues may be applied to the present invention so as to infer information concerning position and orientation of the user while interacting with the present invention. Again, it is preferred that such cues as applied are inferential rather than explicit. An example of a visual cue is the use of language or near-language that within its four corners expresses information concerning position and/or orientation. This might include signs. Visual cues that are more subtle that do not require identical communication between signage and a user are preferred.

A first example of visual cues relates to the designated sanitization area 110. The designated sanitization area, which is determined to be the optimal or near-optimal position of users should be demarcated with a visual indicator 172. The visual indicator 172 may include a visual indication of a lane bearing the designated sanitization area, the boundaries of the same, or the positions beyond the designated sanitization area. By lane, it is meant a demarcation of a pathway corresponding to the designated sanitization area that a user is intended to traverse. These lanes can be single or side-by-side—so long as there is space between the designated sanitization areas for light emission sources to be provided with a clear pathway to sanitize the user.

A visual indicator 172 may extend to the velocity of the user through the device 100. For example, the user's velocity may be tracked with a position indicator 170 that is in signaled communication with lights that convey colors to a user based on the velocity. In one version of the present invention, lights positioned throughout the device interior show a green light to a user as the user proceeds through the device at a rate suitable to provide the exposure dose calculated to be sufficient. If the user proceeds through the device at a rate calculated to exceed the speed necessary to result in the exposure dosage, then yellow lights may be presented to the user; and a red light if the user needs to stop progress altogether. Furthermore progress lights for one set of position and orientation may be used while a second set of lights is used to indicate lateral position and orientation. For example, side lights may be utilized to indicate lateral position, while overhead lights may be utilized to indicate progress/speed.

The present invention can adjust power or exposure time, based on the speed the user is moving through the sanitization area. If the user is moving at a speed that might otherwise result in a shortened exposure time, the machine adjusts 484 intensity while tracking the user to ensure an effective dose has been applied. Additionally, users can be scanned with the light source in conjunction with Artificial Intelligence or camera systems ensuring they have been properly disinfected of any surface based pathogen by moving or steering the light source. The adjustment 484 of the emission source may be any alteration to a characteristic of the emission source that results in an effective dose under the present invention. In other embodiments of the present invention, the adjustment 484 may be the alteration of position of the emission source in order to supply an effective dose based on the position or velocity of a user.

Other examples of visual indications include static, as opposed to dynamic representation of correct user position or velocity, such as icons or pictograms to demonstrate body positions and movement directions. An example is a xray scanner in the airport showing how to properly hold hands and stand in specific marked position.

Physical Structure and Timing

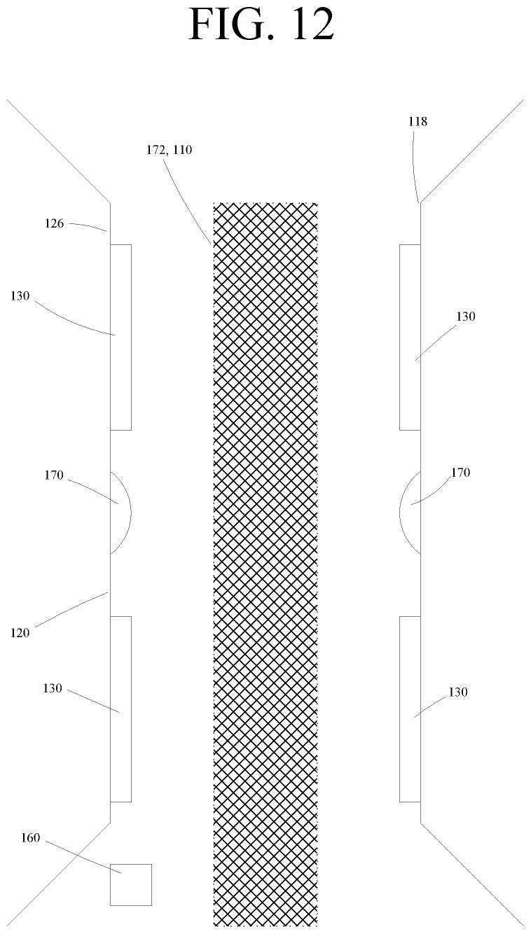

The physical structure of the present invention may be utilized to force an outcome related to exposure period and position and orientation. For example, it is preferred that the present invention utilize a ‘chute’ based entry structure 118 frequently utilized with livestock to ensure that users position themselves sequentially. Light emissions should not be obstructed in the present invention and accordingly users ought not be positioned shoulder-to-shoulder. Light waves should be able to contact users from multiple angles. In the embodiment of the invention in FIG. 12, the chute 118 entryway is used to gain access to the tunnel while an audio source 160 emits inferential audio cues. The visual cue 172 of FIG. 12 includes a pathway marking that corresponds to the designated sanitization area 110. The designated sanitization area 110 is in the middle of the hallway at a middle point between the sidewalls 126. In FIG. 12, the hallway is provided to be a length calculated to be sufficient to provide an acceptable exposure duration period 482 if a person walks at a normal pace. The position indicator 170 can further measure the height of the user in order to signal to the audio source to provide a walking beat or adjustment thereto based on the calculated stride of the user.

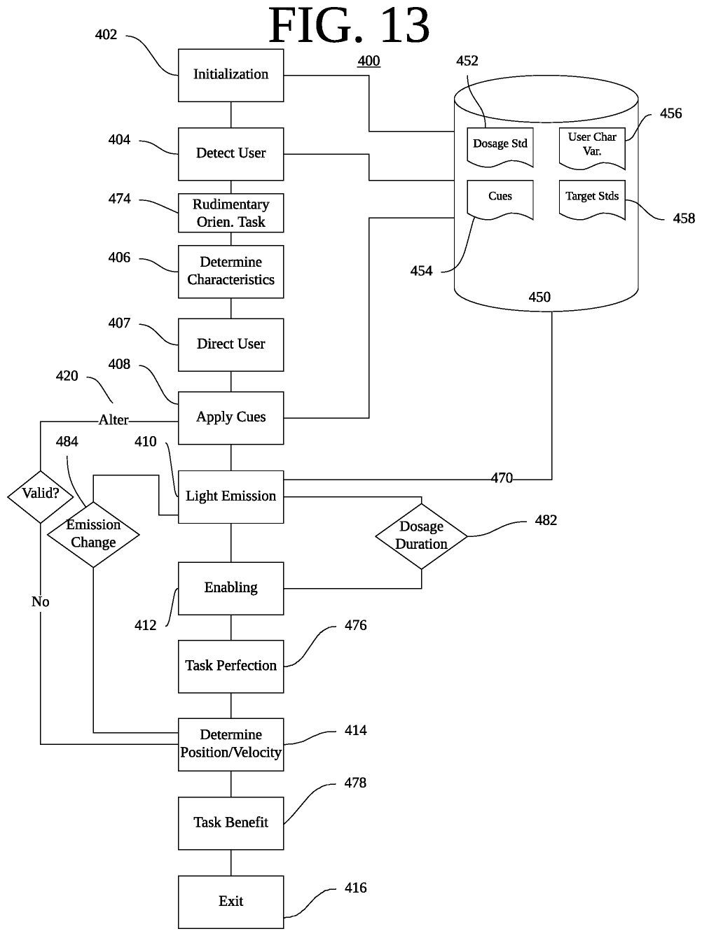

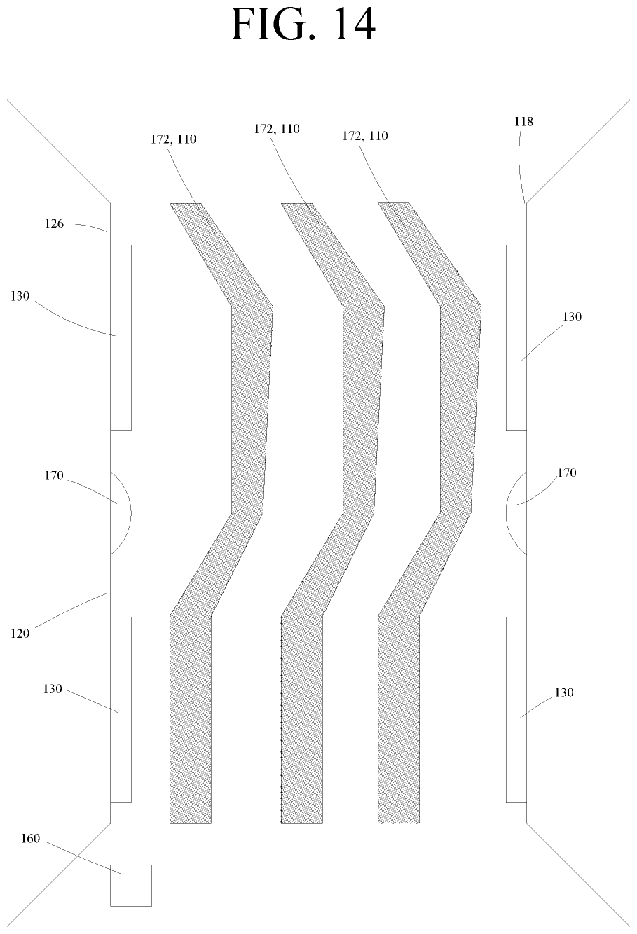

The physical structure of the present invention can also alter the bounded area in which the user 900 may be directed. As shown in FIGS. 13-17, and in particular FIG. 14, a purpose of the present invention is to allow mass sanitization of a public in motion, e.g. airports, theme parks, shopping centers, etc. Therefore, any aspect of the present invention can be multiplied in accordance with this benefit. A bounded area as shown in FIG. 14 includes at least two sidewalls 126 that engulf multiple designated sanitization areas 110, each with visual indications 172 thereof and to guide users on the path best suited to sanitization. One of the complexities in using light sanitization in relation to the public at large is urging them to adopt orientations amenable to proficient light sanitization. The solution is to urge the public on a user-by-user basis to willingly adopt sanitization orientations. One potential solution is shown in FIG. 14, which includes a tortious path laid out as a designated sanitization area 110 and depicted with visual indicators 172. Users shift their orientations and adopt new orientations when presented with non-linear pathways. So even having multiple users in a side-by-side fashion can accomplish the benefits of the present invention, however, the placement of light emissions sources 130 in ceilings in sufficient orientations will require more thought.

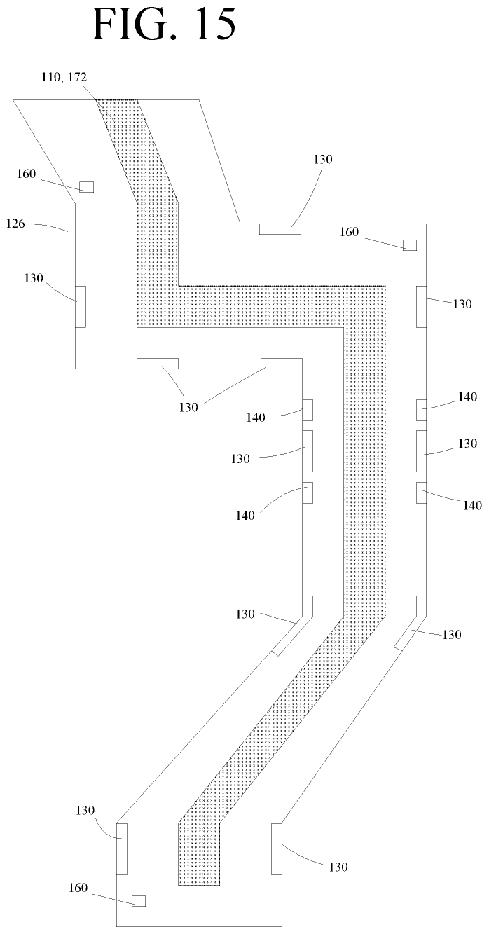

As shown in FIG. 15, the use of non-linear pathways can be examined in a more-thorough fashion. Not only does the designated sanitization area 110 twist and turn, but so does the structure engulfing it. A tunnel with multiple sidewall orientations ensures that the user's body will be exposed to multiple angles of light emission. Each turn of the sanitization area 110 may result in a user adopting a new, different orientation. Each new orientation may expose some portion of the user that was not previously exposed. Twists and turns help ensure that a satisfactory degree of the user's body is exposed to light emissions.

With reference to FIGS. 13-17, the most preferred orientation of a user 900 is a ‘hands-up’ orientation that results in almost the entirely of the human form being available to light emission. Again, a good solution is to urge the user to willingly adopt the ‘hands-up’ position or a position similar thereto wherein arms are lifted to expose the under-arm and side torso. The method 400 of the present invention may include disclosing 474, or perhaps assigning, a user with directions for a predetermined task. The task directions 474 should result in a task perfection 476 period where there is an altered user orientation 920 that allows exposure of the user in orientations other than a rest/relaxation position. Then there is a payoff with some task benefit 478 for the user. For example, in FIG. 16, the user 900 may start off in a natural position and the user is provided with a token as an article 902. For purposes of the present invention, a token includes any material object that a user 900 can manipulate/move in order to assume a secondary orientation 920 designed to differ from a natural, or relaxed, position. With further reference to FIG. 16, the token 902 can be coin that this is provided to a user for insertion into an acceptance machine 492 with an object mark 493. By mark 493 it is meant a target for a user 900 to contact, directly or indirectly with another object (e.g., a token), that allows the user 900 to assume the secondary orientation. A preferred position of the mark 493 is a high position that results in a user 900 assuming as a secondary orientation 920 a ‘hands-up’ orientation. As can be seen in FIG. 16, a user that is handed a coin for insertion into a coin slot in a high position would necessarily be urged to reach with his hand to the highly positioned coin slot to perfect whatever transaction the coin is used. Preferably, there is some form of reward that benefits 478 the user so that the user feels positively compelled to complete the transaction. For an amusement park, for example, the coin may result in reduced waiting in line (commonly referred to as a “Speed Pass”) or admission into the ride. Light emission sources should be placed in the proximity of the mark 493 and machine 492 so that the secondary orientation 920 can be exploited for purposes of sanitization.

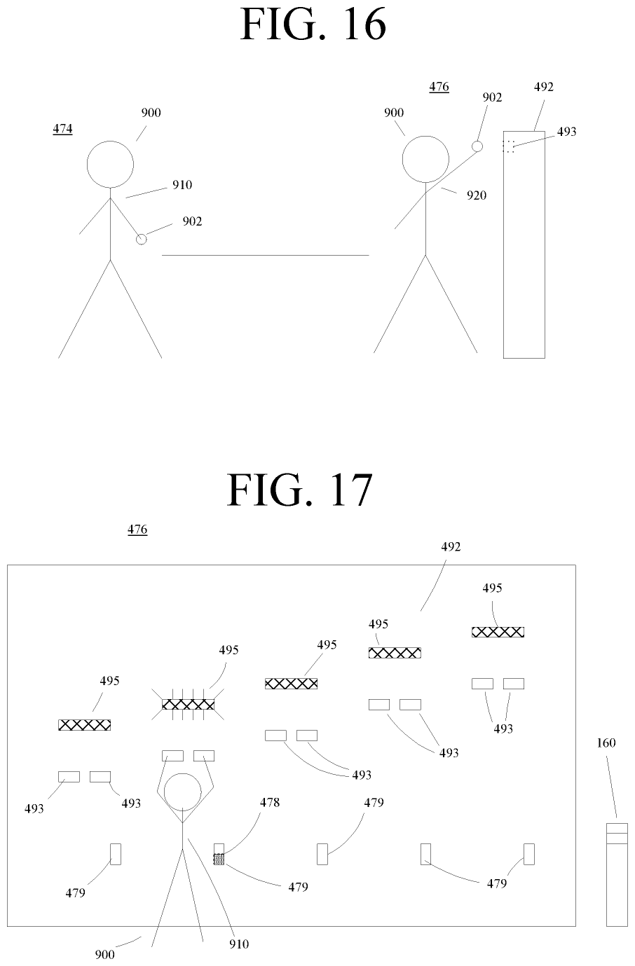

A token is not necessary for purposes of the present invention, merely that a task is presented 474 to the user 900 so that the user can adopt the secondary orientation 920. As can be seen in FIG. 17, the task machine 492 can include multiple marks 493 and sets of marks. Here, the targets 493 can be buttons or other manipulation components that require contact with the user 900 in order to perfect the task 476. Again, the location of the marks 493 should be such that the users assume a secondary orientation 920 that exposes a new portion of the user to light emission. In this version, the machine includes marks 493 that require two hands held above a head for perfection of the task. After the buttons are pressed, perhaps required to be pressed simultaneously, a task benefit 478 is issued. The benefits 478 can be acquired via a task benefit dispenser that provides the benefit upon completion of the task. Note that in FIG. 17, the machine 476 include multiple marks and mark sets. The significance of the multiple sets is that orientations may change based on the position and velocity of the user, and for purposes of the present invention, position also includes all areas of mass for a user body and indications of the same (e.g., height and girth). Accordingly, a detector 160 can be positioned proximate to the machine 492 such that a height of the user is determined, and a mark set that results in the preferred orientation is indicated via the task indicator 495, here, a blinking light. Contacting the marks that are not indicated do not result in perfection of the task. Children and adult, or the physically handicapped, may each have their own target set such that the taller users act upon higher marks than shorter users for perfection of the task.

Targets of the present invention are pathogens and hazardous (or other unwanted) chemicals. Pathogens that may be targets of the present invention include: Viruses, Bacteria, mold, and Fungi can survive on human skin, clothing, and air long enough to be transferred to another human or compromise the immune system. These organisms can be destroyed when exposed to Far UVC light for a certain time. Other targets may include DNA/RNA Fragments, viruses, bacteria, mold, fungi, pollen/allergens, mycoplasmas, amoebas, sperm, and miscellaneous single cell organisms. Chemicals that may be targets of the present invention include: Benzene, Formaldehyde, Organophosphate, nerve agents, hydrocarbon, fluorocarbon or chlorocarbon and sulfur containing compound such as thiols and mercaptans

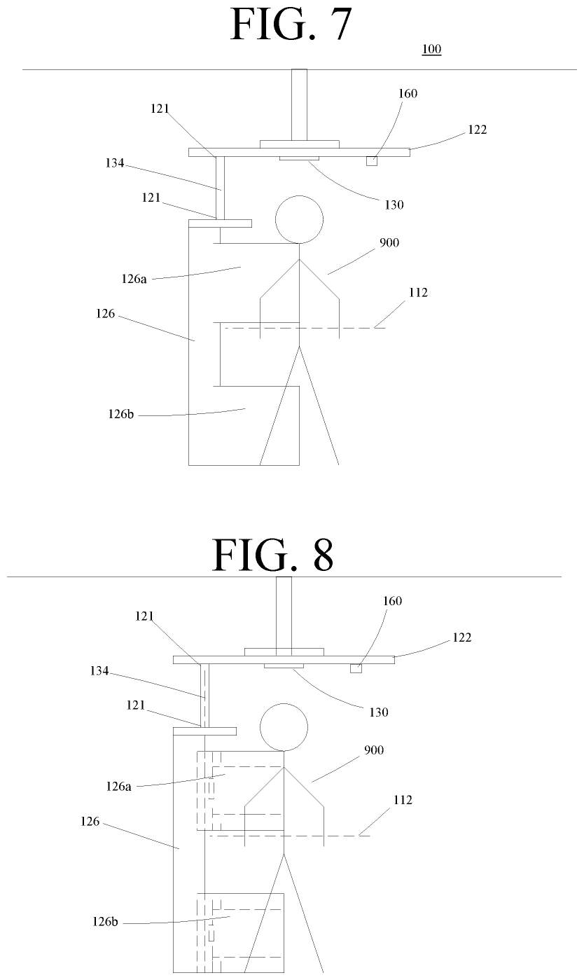

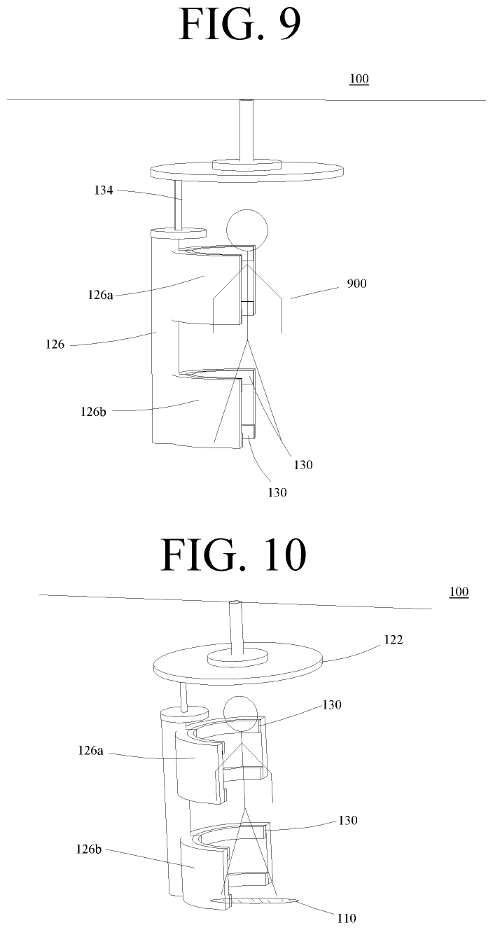

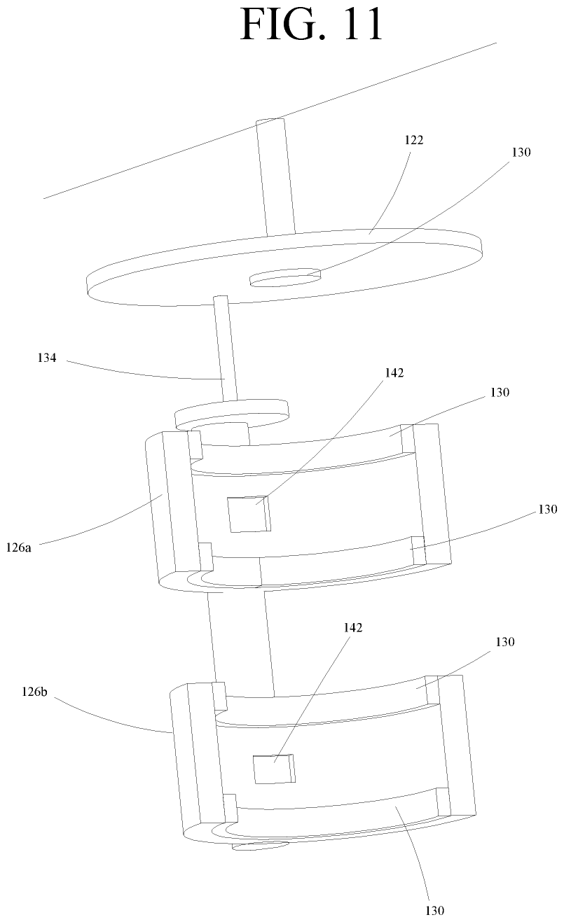

In FIGS. 1-6 exposure is at least partially controlled by the pace of the user through the device 100. Turning now to FIGS. 7-11, the present invention 100 may also include embodiments that work in conjunction with a relatively stationary user 900. Rather than surrounding the user with the device and its operable components, the device 100 actuates in relation to the user. The Actuating Embodiment 100 of FIGS. 7-11 may include a topwall 122 that bears at least one stationary light emission source 130 positioned above the user. The device 100 includes an arm 134 that rotates about the topwall 122 about a track (not shown) that completes a circle or near circle about the user 900. The arm 134 connects to a sidewall 126 that manifests in an upper branch 126a and a lower branch 126b. The device includes movement points 121 at the connection between the arm 134 and the topwall 122 as well as the connection point between the arm 134 and the sidewall 126. The movable connection 121 between the topwall 122 and the arm 134 is meant to travel about the periphery of the version of the topwall 122 as shown such that the arm travels in a circle carrying the sidewall circularly about the user. The connection 121 between the arm 134 and the sidewall 126 is a rotatable connection that allows the sidewall to spin in relation to the arm so that as the sidewall 126 moves around the user that the sidewall also rotates around the user

The sidewall 126 includes sanitization components of the present invention, including the disinfectant enabling devices 140, here once again manifested as environmental agitation devices. In the environmental agitation devices 140 of the actuating version of the device, it may be advantageous to utilize sonic motivators that rather than relying on physical disturbance of the air surrounding the user, rely on sonic vibration to agitate the articles of clothing and other articles of the user. One series of tests conducted by James Larson on Bass: the Physical Sensation of Sound in the Audioholics online A/V Magazine, conducted with tones from 31-50 Hz at 90-100 dB output levels compared how people perceived their sensations versus actual vibration levels of different areas of their body by hooking up accelerometers to the head, abdomen, and chest of their test subjects. It was found that although the head itself was not measured to vibrate as much as the abdomen and chest, head vibrations were perceived as being stronger, likely due to auditory structures within the head. Chest vibrations were measured and subjectively felt to be stronger than abdominal vibrations, and 50 Hz frequencies were more effective at causing vibrations and vibratory sensations than lower frequency sound at the same output level. Other tests conducted at much more powerful levels past 140 dB reported respiratory rhythm changes, gagging, chest wall vibrations, and perceptible visual field vibration. By the use of “sonic” in the present disclosure, it is meant the application of sound of any type/intensity/level to physically disturb an environment, and is not meant to include connotations related to whether the human ear can detect such sound.

In addition to utilize speakers to create vibrations, vibrations can be mechanically created by vibrating all or part of the designated sanitization area 110, shown here as a visual indication 172 of the region in which the user should stand. The designation sanitization area 110 can be fabricated to be a discrete platform connected to the remainder of the device 100 such that the area stand 110 can motion independent of the remainder of the device.

The actuating version 110 of the present invention relies on light emission sources above and below the medial line 112, which can generically determined or determined on a case-by-case basis by a position indicator 160. The light emission sources 130 provide a dosage commensurate with the predetermined, or determined on a case-by-case basis, dosage amount for the dosage period. Agitation occurs during the dosage period to ensure a high degree of cleansing. Here, because the agitators 140 as shown will be rotated about the user, there is not a need to have a second agitator at a counter-position, which brings up an important point concerning the agitation in multiple directions: the multiple directions do not need to occur simultaneously.

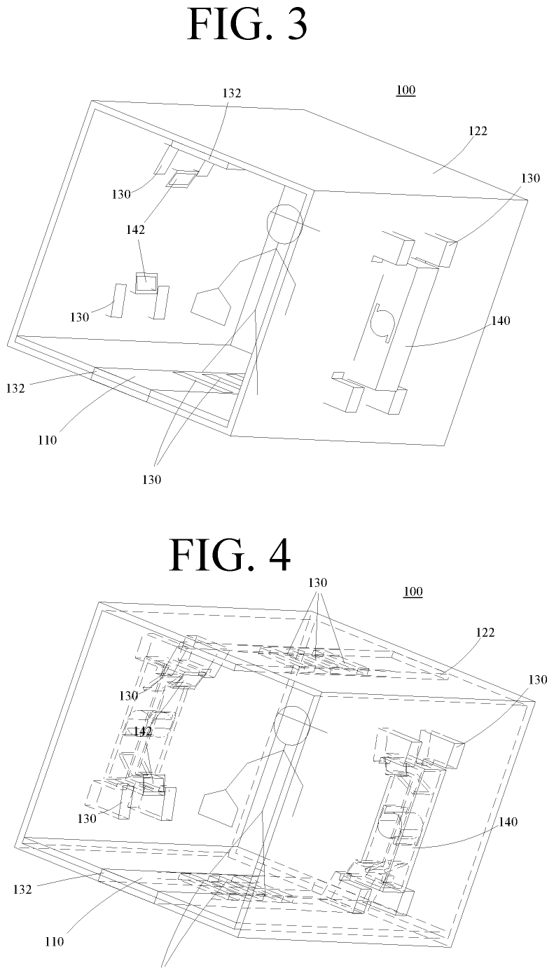

Returning to FIGS. 2-6, actuating features of the present invention can apply to any embodiment thereof. With specific reference to FIGS. 3-5, the present invention can utilize floor light sources 130 that are applied to a transparent floor portion 124 corresponding to the designated sanitization area. The designated sanitization area 110 may be actuating similar to that of a moving stairway or moving floor. The light sources 130 are positioned on a light track 132. Such an arrangement may similarly be utilized on ceilings as well so that the light emission sources 130 motion with the user 900.

Turning now to FIG. 13, the present invention includes a method of sanitization 400. The method of sanitization includes directing 407 the user into a designated sanitization area having at least one light emission source adapted to generate an electromagnetic frequency of light effective for order-of-magnitude destruction of predetermined pathogens for a dose duration positioned both above and below a medial point. Then the light emission source is motioned 410 in relation to the user (i.e., the user moves, the light source moves, or both move in concert such that relative positions are altered) such that the light emission source accesses both the fore and aft of the user. The method applies 408 an inferential indication cue to the user based on a determination at least one of a position and a velocity of the user. Furthermore, the method may apply disinfectant enabling 412 that alters the environment in/on which the light acts as a sanitization agent.

The method 400 includes initialization 402 whereby the components of the present invention are made ready to engage in the method. This can be such rudimentary steps as engaging the computer controllers that operate the device and method, initiating alternating or direct current power to the components, accessing formulae for exposure, timing, etc. The method 400 may include as an initial step detecting 404 a user upon which the device and method will act. The detection 404 may simply include recognizing the existence of a mass in the proximity of the device, or in versions of greater complexity determining 406 the characteristics of the user. Any characteristic of a user that can be perceived by a machine may be of use to the present invention. Characteristics determinations of consequence to the present invention include radar evaluations of height and/or girth, boundary detection to determine the existence of articles on the user (e.g., sunglasses, hats, purses,), thermal imaging of users to determine physical characteristics, etc.

The user is then directed 407 into the device 100. The direction of the present invention may include such simplistic principles as indicating (presumably, by simply not barring) the entrance to a bounded tunnel or indicating a designated sanitization area for the Actuating Version of the present invention to apply light emission. As the user nears or enters the present invention, cues are applied 408 to aid the user in maximizing the benefits of light sanitization. Audio and visual cues, or a combination thereof, may be applied as discussed elsewhere in the present disclosure. Light is emitted 410 pursuant to the dosage duration intended to supply the maximum benefit with the least amount of feasible exposure. During the light emission 410, sanitization enablers apply 412 secondary stimulus to the user or its nearby environment to enable the light sanitization. During this period, present invention may periodically, constantly, or otherwise determine the position and/or velocity of user(s) in the device. Such measurements of position and velocity have the uses discussed elsewhere in this disclosure; however, updates to the position and velocity allow the user to alter 420 the cues so as to provide the dose commensurate with pathogen/chemical destruction efficacy. Finally, the user exits 416 the device such that no further cleansing occurs.

Prior to, during, and after the light emission of the present invention, the method and systems may access memory resources 450 related to the destruction of pathogens and chemicals. The present invention can include a target dossier file 458 that includes data related to pathogens and chemicals that may be the target of destruction, modification, denaturization, etc. The dossier files may include in particular such data as spectrum characteristics related to destruction, modification, and denaturing of the pathogen/chemical. Such actions are not necessarily the same; it may be the case that modification is preferred to destruction. It is preferred that this data is used to “tune” 470 the light emission sources to the one or more wavelengths for the effect desired on the chemical/pathogen. In embodiments of the present invention. For example, a construction site may result in exposure to CHEMICAL A and PATHOGEN B. If it is known that two different spectra are required for each of these entities, then the light emission sources can be instructed to apply the spectra necessary for the dosage duration for each of the entities. The light emission sources may flash between the spectra, or may entirely apply one spectrum prior to ‘hopping’ to the next spectrum. Furthermore, the likely location of target entities may be of consequence such that light emission can be tuned to a particular coordinate system. In other words, because E. coli is most likely to be acquired at the feet of a user, and users may be wearing shoes when utilizing the method of the present invention, the light emission sources oriented toward the feet of a user may apply a different dosage duration and/or intensity than other parts of the body. Because shoes include materials that shield the body from many light spectra, an intensity considered “harsher” to the human body may be applied at these lower regions.

Dosage standards files 452 may be maintained on the spectra and intensity associated with the light emission sources. Certain materials may be more or less impervious than others to particular spectra and as articles are detected 406 that affect spectra, the spectra may be adjusted.

Cue files 454 may be maintained in the storage memory 450 of the present invention such that cues can be provided on a case-by-case basis per person. For example, a cue can be as discussed to include the walking stride of an average person. However, the stride of an average person may vary widely based on physical characteristics such as height and weight, or based on culture. The cue files 454 may include various average strides calculated by sex, height, weight, ethnicity, and culture (principally determined by location of the device). Any variation of a cue based on the physical, cultural, or other characteristics of the users/geography can be applied to the method.

User characteristic variables 456 may be utilized to vary the application of the present invention based on user specific traits. These user specific traits may be based on inherent recognition of a user as JOHN DOE, who is known to have sensitivity to FAR UVC light, or because of recent medical treatments cannot undergo further exposure to UVC light. Otherwise, variables based on non-personal data, e.g. height, age, weight, hair color, hair type, etc. may be applied or changed based on user characteristic determination.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. An electromagnetic spectrum disinfectant device comprising:
a bounded tunnel having a centralized focal point;
a light source adapted to emit an electromagnetic frequency of light effective for order-of-magnitude destruction of predetermined pathogens for a dose duration, within said tunnel, positioned both above and below a pre-determined bifurcation of said bounded tunnel oriented towards a tunnel medial point; and
an environmental agitation unit, oriented toward said centralized focal point, adapted to physically administer atmospheric disturbance proximate to said focal point.

2. The device of claim 1 further comprising an audio source adapted to provide an inferential indication cue related to a user position within said tunnel.

3. The device of claim 1 further comprising an audio source adapted to provide an inferential indication cue related to a user velocity within said tunnel.

4. The device of claim 1 wherein said inferential indication cue includes rhythmic repetition adapted to correspond to steps of a walking velocity.

5. The device of claim 1 further where said dose duration is a pre-determined dosage duration, and wherein a length of said tunnel is dimensioned to apply said electromagnetic frequency to a user for a predetermined time period based on a predetermined user velocity based on a location-placement of said device,

6. A method of electromagnetic sanitization for a user, said method comprising:
directing the user into a designated sanitization area having at least one light emission source adapted to generate an electromagnetic frequency of light effective for order-of-magnitude destruction of predetermined pathogens for a dose duration positioned both above and below a medial point;
motioning said at least one light emission source fore and aft said user; and
providing an inferential indication cue to the user based on a determination at least one of a position and a velocity of the user.

7. The method of claim 6 further comprising altering said inferential indication cue based on said user position/velocity determination.

8. The method of claim 7 wherein said altering step includes a visual indication related to speed.

9. The method of claim 7 wherein said altering step includes a visual indication related to lateral position.

10. The method of claim 7 wherein said altering step includes a visual indication related to user proximity.

11. The method of claim 7 wherein said alteration step includes an audio indication related to user stride.

12. The method of claim 7 wherein said alteration step includes an audio indication related to lateral position.

13. The method of claim 6 further comprising measuring a physical characteristic of the user and selecting said inferential indication cue based on said physical characteristic.

14. The method of claim 6 further comprising physically agitating an environment surrounding said user within said predetermined sanitization area.

15. The method of claim 6 further comprising sonically agitating an environment surrounding said user within said predetermined sanitization area.

16. The method of claim 6 further comprising modifying a dose intensity based on at least one indication of position and velocity of said user.

17. The method of claim 6 wherein said motioning step includes positioning a first light emission source below said median line and positioning a second light emission source above said medial line.

18. A method of electromagnetic sanitization for a user, said method comprising:
directing the user into a designated sanitization area having at least one light emission source adapted to generate an electromagnetic frequency of light effective for order-of-magnitude destruction of predetermined pathogens for a dose duration positioned both above and below a medial point;
motioning said at least one light emission source fore and aft said user; and
physically agitating an environment surrounding the user within said predetermined sanitization area.

19. The method of claim 18 wherein said agitation includes bidirectional agitation.

20. The method of claim 19 wherein said agitation includes fluid flow agitation.
